(19) United States
(12) Patent Application Publication
(10) Pub. No.: US 2025/0339330 A1
(43) Pub. Date: Nov. 6, 2025

(54) New Crutch System

(71) Applicant: Daryl Dion Moody, INDIANAPOLIS, IN (US)

(72) Inventor: Daryl Dion Moody, INDIANAPOLIS, IN (US)

(21) Appl. No.: 19/267,284

(22) Filed: Jul. 11, 2025

Related U.S. Application Data
(63) Continuation-in-part of application No. 16/557,568, filed on Aug. 30, 2019, now abandoned.
(60) Provisional application No. 62/738,545, filed on Sep. 28, 2018.

(51) Int. Cl. A61H 3/02; A61F 5/058

(52) U.S. Cl. CPC A61H 3/02; A61F 5/0585; A61H 2003/0233; A61H 2201/105; A61H 2201/5007; A61H 2201/5025

(57) ABSTRACT

A crutch system designed as a walking aid that transforms into various forms, including a crutch and cane, allows for significant time savings by eliminating multiple visits to medical facilities for length adjustments during recovery. The system features an “L” shaped leg stabilizer that keeps the injured leg straight, with adjustable straps for securing the foot and leg to facilitate healing. A telescopic walking-aid apparatus allows height adjustments and supports the user under the armpit or in hand, with multiple handles for added stability. An adjustable angle hinge secures the thigh and knee plates to prevent unwanted movement while accommodating various injuries. Additionally, a protective knee brace includes ventilation for comfort and interlocking fasteners for comprehensive coverage. The design incorporates spring-loaded locking mechanisms for easy length modifications throughout the recovery process, ensuring adaptability to user needs.

### FIELD OF THE INVENTION

The present invention generally relates to a new crutch system. More specifically, the present invention is a combination of a knee brace, an adjustable walking-aid apparatus, a leg stabilizer, and a plurality of stabilizer straps.

### BACKGROUND OF THE INVENTION

Leg injuries, ranging from minor sprains to severe fractures, often result in extended recovery times during which patients must rely on various mobility aids to navigate their daily lives. These walking aids, which include crutches, canes, and walkers, play a crucial role in helping patients maintain mobility while minimizing weight-bearing on the injured leg. However, the current solutions have notable limitations.

One significant issue is the necessity for frequent adjustments to these walking aids as the patient's condition evolves. For instance, crutches often require multiple length adjustments throughout the healing process, leading to not only logistical challenges but also increased healthcare costs associated with frequent medical visits. Patients may incur additional expenses for professional fittings and new equipment, which can be both inconvenient and burdensome. Moreover, many walking aids provided by medical personnel lack the necessary adjustability, which can result in further complications or injuries, such as improper alignment or lack of support, exacerbating the original condition beyond the recovery period.

The existing walking aids can also create difficulties in daily activities and impede a patient's independence—an essential aspect of quality of life during the recovery phase. Patients may find themselves limited in their ability to engage in routine tasks, leading to frustration and a slower recovery process.

Thus, there is a need to develop a device or system that addresses these challenges by providing a comprehensive, customizable solution for walking assistance that adjusts seamlessly to the patient's height, weight, and stage of recovery. The present invention aims to fulfill this need by offering a combination of a customizable walking-aid accessory, a knee brace, a leg stabilizer, and a plurality of stabilizer straps. This integrated system not only allows for easy adjustments without the necessity of multiple medical appointments but also ensures that the injured limb is adequately supported, promoting a more stable and efficient recovery process. The present invention is applicable to various leg injuries, including but not limited to, sprains, strains, total knee replacements, ligament injuries, fractures, and Achilles tendon injuries. By maintaining the injured limb in a stable position, this innovation aims to significantly reduce recovery time and improve overall patient outcomes.

The present invention is intended to solve the problems associated with conventional devices and methods and provide improvements on these devices.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

A walking aid system designed to support individuals with injuries or mobility impairments comprises a leg stabilizer, a knee brace, and a walking-aid apparatus that can be adjusted for optimal functionality and user comfort.

The leg stabilizer is an “L”-shaped component configured to maintain a user's leg in a straight or bent position. It includes an adjustable angle hinge, a plurality of medial strap openings, and lateral strap openings, as well as stabilizer straps constructed from durable materials, which function to secure the user's leg effectively. This design facilitates the healing of fractured bones and aids in recovery from various leg conditions.

The walking-aid apparatus is characterized by a height-adjustable telescopic structure, comprising an upper support assembly, a first handle, a second handle, and a lower support converging to a singular ground-contacting point. This apparatus permits adaptable use by positioning under the user's armpit or being held in hand, thereby providing essential support for the injured leg.

The knee brace is specifically designed to protect the anterior, medial, and lateral aspects of the knee and incorporates multiple strap openings to ensure secure attachment. In certain embodiments, the knee brace includes integrated medicine tubes arranged around a ventilation hole, connected to a medicine reservoir for dispensing medicinal fluids. Additionally, a remote controller enables users to conveniently manage the dispensing of these fluids.

Furthermore, the walking aid system encompasses a telescopic panel assembly attached between the pair of lower supports, allowing for length modifications throughout the patient's recovery process. An adjustable locking mechanism is also incorporated to secure various positions and heights within the system. Enhanced stability and comfort are achieved through the inclusion of neoprene cushioning and ambidextrous handle placements.

This innovative system effectively addresses the challenges encountered by individuals requiring walking assistance, reducing dependency on multiple medical visits for adjustments and significantly enhancing the overall user experience during the recovery process.

### BRIEF DESCRIPTION OF THE DRAWINGS

FIG. 1: Illustration of one embodiment of the walking aid system of the present invention.

FIG. 2: Perspective view of the walking-aid apparatus.

FIG. 3: Perspective view of the leg stabilizer.

FIG. 4: Exploded view of one embodiment of the adjustable hinge featuring a push-button mechanism.

FIG. 5: Front view of the leg stabilizer, highlighting the plurality of medial strap openings and lateral strap openings.

FIG. 6: Perspective view of the leg stabilizer with stabilizer straps looped through the medial strap openings and lateral strap openings.

FIG. 7: Front perspective view of the knee brace, illustrating the arrangement of strap openings.

FIG. 8: Back perspective view of the knee brace, showing the brace straps inserted through the strap openings.

FIG. 9: Front perspective view of the knee brace, depicting the plurality of medicine tubes and the associated reservoir.

FIG. 10: Illustration demonstrating the detachability feature of the walking-aid apparatus.

FIG. 11: Illustration of the telescopic panel assembly of the walking aid system.

FIG. 12: Illustration of the telescopic panel assembly featuring the first handle.

### DETAIL DESCRIPTIONS OF THE INVENTION

All illustrations of the drawings are for the purpose of describing selected versions of the present invention and are not intended to limit the scope of the present invention.

As a preliminary matter, it will be evident to those with ordinary skill in the relevant field that the present disclosure has significant utility and application. Any embodiment may incorporate one or multiple aspects disclosed herein and may further integrate any combination of features mentioned above. It is important to note that any embodiment referred to as “preferred” is considered a best mode of implementing the embodiments of this disclosure. Other embodiments may also be explored for illustrative purposes to provide a full and enabling disclosure. Additionally, many adaptations, variations, modifications, and equivalent arrangements will be implicitly included within the scope of the embodiments described here.

Accordingly, while the embodiments are presented in detail concerning one or more variations, it should be understood that this disclosure serves as an illustrative and exemplary representation of the present invention and is intended solely to facilitate a full and enabling disclosure. The detailed presentation of one or more embodiments is not designed to limit the scope of patent protection afforded by any claim that may issue from this disclosure; that scope will be defined by the claims and their equivalents. It is not intended for the scope of patent protection to be constrained by any claim limitation found herein or in any resulting patent that does not explicitly appear in the claim itself.

For instance, any sequences or temporal orders of steps in the processes or methods described here are intended to be illustrative and not restrictive. Thus, while various processes may be depicted and described in a particular sequence or order, it should be understood that these steps are not confined to any specific arrangement unless specifically indicated otherwise. Indeed, the steps of these processes or methods may be executed in various sequences while still falling within the scope of the present disclosure. Therefore, it is intended that the scope of patent protection be determined by the issued claims rather than the descriptions provided here.

Furthermore, it is essential to recognize that each term used in this disclosure conveys that which an ordinary artisan would understand. If a term's meaning deviates from a particular dictionary definition due to the contextual use in this document, the interpretation by an ordinary artisan will take precedence.

In addition, the terms “a” and “an” generally denote “at least one,” implying that a plurality is also possible unless the context specifically dictates otherwise. When used to connect a list of items, “or” indicates the existence of at least one item from the list without excluding multiple items. Conversely, “and” signifies all items in the list.

The subsequent detailed description refers to the accompanying drawings, where the same reference numbers are consistently applied across both the drawings and the description to denote similar elements. While numerous embodiments of the disclosure will be described, modifications, adaptations, and various implementations are feasible. This includes potential substitutions, additions, or modifications to the illustrated elements, as well as alterations in the order of or the inclusion of steps in the described methods. Hence, the detailed description that follows does not impose limitations on the disclosure. Instead, the proper breadth of the disclosure is defined by the claims contained herein and/or those that may be issued.

The present disclosure encompasses various aspects and features. While many of these aspects and features are framed within methods, systems, apparatuses, and devices for providing a comprehensive, customizable solution for walking assistance that adjusts seamlessly to the patient's height, weight, and stage of recovery, it should be understood that the embodiments of the present disclosure are not restricted solely to this context.

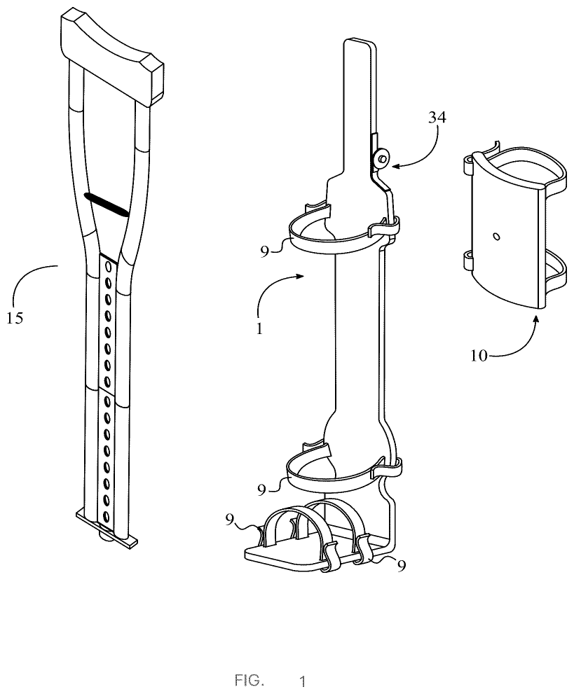

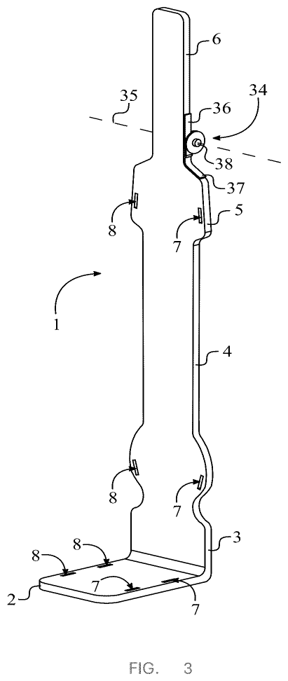

Referring to FIG. 1 and FIG. 3, the present invention pertains to a novel crutch system designed as a versatile walking aid that can be transformed into a crutch, cane, or other walking devices. This invention significantly reduces the time and effort expended by users, as it eliminates the need for multiple visits to medical facilities for length modifications of the crutch system during recovery.

As shown in FIG. 3, the present invention comprises a leg stabilizer 1, a plurality of stabilizer straps 9, a knee brace 10, and a walking-aid apparatus 15.

In certain embodiments, the plurality of stabilizer straps 9 is fabricated from nylon or another durable material, specifically configured to secure the user's injured foot and leg to the leg stabilizer 1. The leg stabilizer 1 is characterized as an “L” shaped component forming a 90-degree angle, designed to maintain the user's leg in a straight position while ensuring the foot and heel are positioned at a 90-degree angle. This configuration facilitates the healing process for fractured bones or other leg conditions. The leg stabilizer 1 comprises a plurality of medial strap openings 7 and lateral strap openings 8. The medial strap openings 7 are positioned along the medial side of the user's leg and are specifically configured to engage with the plurality of stabilizer straps 9. Likewise, the lateral strap openings 8 are located on the lateral side of the user's leg, also designed for engagement with the plurality of stabilizer straps 9.

Each stabilizer strap 9 is looped through a corresponding medial strap opening 7 and lateral strap opening 8. The user introduces each stabilizer strap 9 through one medial strap opening 7 and subsequently loops it around the front of the leg, securing one end of each strap. The user repeats this process for each of the lateral strap openings 8, thus securing the opposite end of each stabilizer strap 9.

The leg stabilizer 1 is spring loaded to assist a patient comfortability and stability while using. Furthermore, it is also left and right foot molded.

The invention is unique because it is a 3-in-1 combination kit: it comprises a detachable crutch, a leg stabilizer that can be used as a drop foot splint, and also a patella knee brace that alleviates pain for neuropathy.

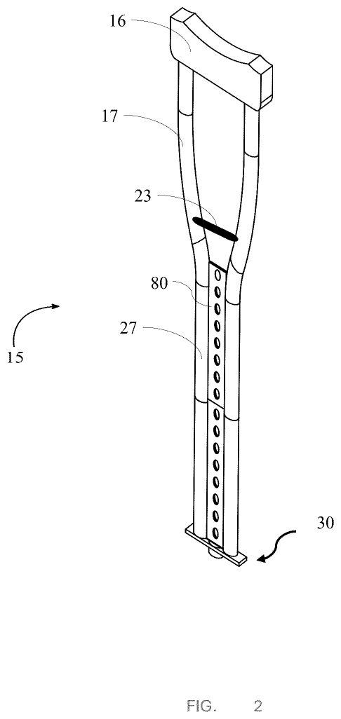

In reference to FIG. 2, the walking-aid apparatus 15 is a device that incorporates a telescopic mechanism, thereby providing a transformable and height-adjustable aid. The apparatus 15 may be positioned under the user's armpit or held in hand to support the injured leg. The walking-aid apparatus 15 includes a first handle 16, a pair of upper supports 17, a second handle 23, and a pair of lower supports 27. A telescopic panel assembly 80 is attached between the pair of lower supports 27.

In one embodiment, the first handle 16 is terminally connected to the upper support assembly 17. The user may either place the first handle 16 under the armpit or grip it in hand to support the injured leg. Some embodiments of the first handle 16 feature a neoprene pad, which provides cushioning when positioned under the armpit. The second handle 23 is also connected to the upper support assembly 17 and is offset from the first handle 16. When the first handle 16 is placed under the armpit, the user may also utilize the second handle 23 for additional support. The lower support 27 is adjacently connected to the upper support assembly 17, opposite the first handle 16, converging to form a singular ground-contacting point.

As referenced in FIG. 3 to FIG. 6, the leg stabilizer 1 comprises an adjustable angle hinge 34, a foot plate 2, a heel plate 3, a calf plate 4, a knee plate 5, and a thigh plate 6. The foot plate 2 is positioned perpendicularly to the heel plate 3 and is adjacently connected. The user places the injured foot on the foot plate 2 with the heel against the heel plate 3 to maintain a perpendicular orientation. The calf plate 4 is terminally connected to the heel plate 3, opposite the foot plate 2. After securing the heel against the heel plate 3, the user ensures the calf fully contacts the calf plate 4 to provide adequate support. The knee plate 5 is terminally connected to the calf plate 4, opposite the heel plate 3, allowing the user to place the knee against the knee plate 5 to keep the joint straight. The thigh plate 6 connects to the knee plate 5, opposite the calf plate 4, via the adjustable angle hinge 34. The user may modify the angle between the thigh plate 6 and the knee plate 5 using the adjustable angle hinge 34 to accommodate various strapping angles of the injured leg.

The straight configuration of the leg stabilizer 1 prevents extensor lag syndrome by maintaining the leg in a stable position during movement. The user may adjust the angle between the thigh plate 6 and the knee plate 5 to cater to different injuries or circumstances as required.

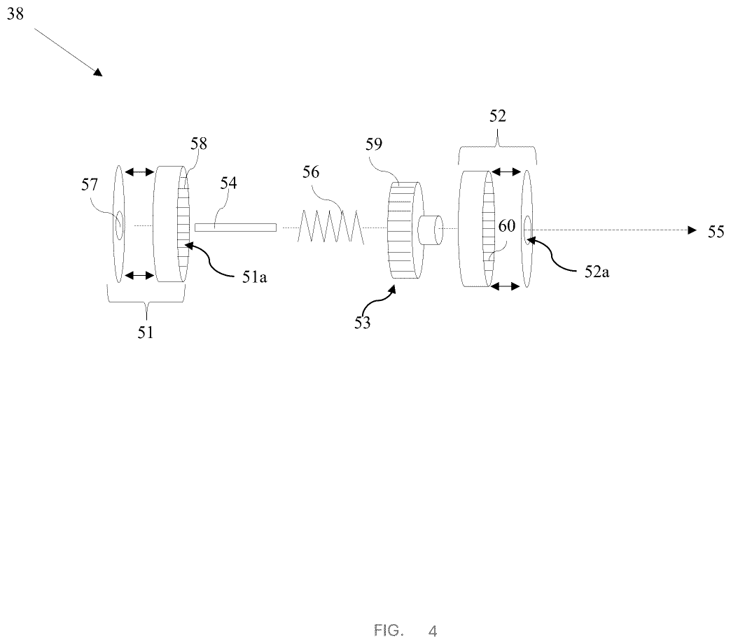

Referring to FIG. 3 and FIG. 4, the adjustable angle hinge 34 comprises a rotational axis 35, a first arm 36, a second arm 37, and a hinge lock 38. The first arm 36 and second arm 37 are rotatably engaged about the rotational axis 35. The user can modify the angle between the first arm 36 and second arm 37 by rotating either arm. The first arm 36 is integrated onto the thigh plate 6, while the second arm 37 connects to the knee plate 5. The hinge lock 38, integrated into both arms, enables the user to secure the selected angle between them.

In one embodiment, the hinge lock 38 comprises a gear mechanism and a cam path facilitating a push-button locking system. As illustrated in FIG. 4, the hinge lock 38 includes a base element 51, a cap element 52, and a central longitudinal axis 55. The first arm 36 is integrally attached to the base element 51, while the cap element 52 is connected to the second arm 37.

The base element 51 can accommodate a resilient spring 56 and includes a cylindrical length characterized by external and internal surfaces, having a cylindrical recess 57. A series of uniform cogs 58 is evenly distributed along the internal side of the base element 51 from the open end, each cog being equidistant and parallel to the central longitudinal axis 55.

The base element cog 58 is designed to receive the corresponding cogs 59 of the push-button element along the cylindrical length at its open end. An aperture 51a is centered on the base element 51, facilitating the inclusion of a push-button rod 54 extending along the central longitudinal axis 55 within the resilient spring 56.

In specific embodiments, the push-button rod 54 is connected to a cam path allowing for stable engagement with the push button 53, ensuring precise activation and feedback. The cam path defines the motion trajectory converting rotary motion into linear motion. The push-button 53 features a cylindrical body with external and internal sides and attaches to the push-button rod 54. The body incorporates a series of cogs 59 spaced evenly along its external surface. Some designs of the push-button 53 may also feature an internal recess to accommodate a resilient spring.

The cap element 52 includes an opening 53a sized for the push-button 53 and features a cylindrical length that connects with the open end of the base element 51. Cogs 58 are uniformly spaced along the internal side of the cap element 52.

The push-button can engage in a locking feature when in the neutral position, wherein the cogs 59 engage both the base element 51 and the cap element 52, thereby preventing rotational movement about the central longitudinal axis 55. Additionally, the push-button 53 may allow for a 360-degree pivot feature between the cap element 52 and the base element 51, enabling transverse rotation about the central longitudinal axis 55 when pressed inward to disengage the cogs, thus unlocking the push-button. Upon reverting to the neutral position, the resilient spring 56 facilitates re-engagement of the cogs 59, securing the adjusted pivoted position.

When the user secures the injured leg to the leg stabilizer 1 and adjusts the first arm 36 and second arm 37 to a desired angle, the hinge lock 38 secures the angle between the thigh plate 6 and knee plate 5, preventing unwanted movement within the leg stabilizer 1.

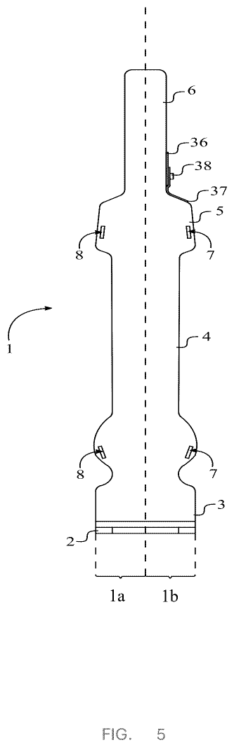

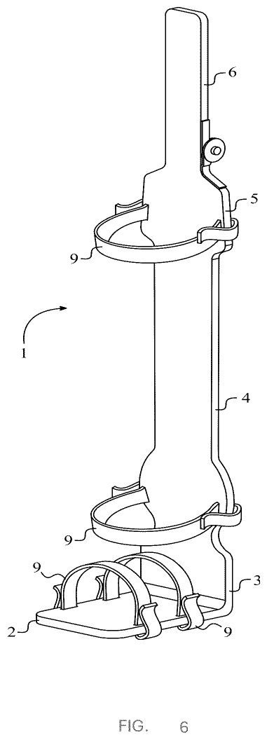

As referenced in FIG. 5 and FIG. 6, the medial strap openings 7 extend through the medial side 1b of the leg stabilizer 1, enabling the user to insert the stabilizer straps 9 to secure the leg to the medial side 1b. Similarly, the lateral strap openings 8 are positioned through the lateral side 1a of the leg stabilizer 1, allowing for stabilization of the leg on the lateral side 1a.

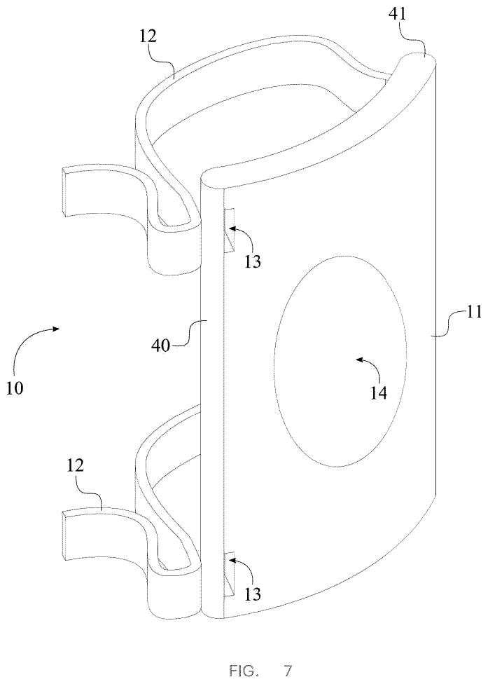

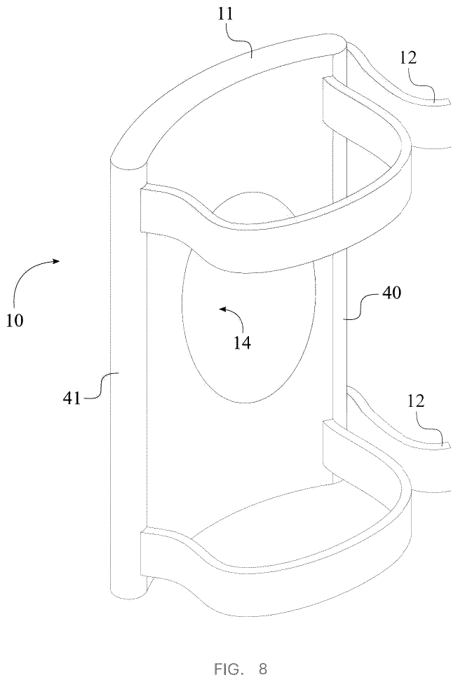

In reference to FIG. 7 and FIG. 8, the knee brace 10 is designed to protect the anterior aspect of the knee, comprising a brace body 11, a plurality of brace straps 12, a plurality of strap openings 13, and at least one ventilation hole 14. The strap openings 13 are linearly arranged along the first edge 40 of the brace body 11 and are configured for the introduction of the brace straps 12 to secure the brace body 11 to the anterior knee. The brace straps 12 are attached to the opposing edge 40 of the brace body 11.

To secure the knee brace 10, the user positions it at the front of the knee, pulls the free ends of the brace straps 12 around the back of the knee, and introduces them into the strap openings 13 for proper fastening.

In another embodiment, the knee brace 10 extends coverage from the front of the knee to both the medial and lateral sides. Furthermore, the present invention may include at least one medial attachment clip and at least one lateral attachment clip, each featuring a first and second interlocking fastener. The first interlocking fastener is connected to the brace body 11, while the second fastener is affixed to the knee plate 5. Once the injured leg is secured to the leg stabilizer 1, the user places the knee brace 10 atop the knee, interlocking the fasteners of both clips to create comprehensive coverage of the knee area, integrating with the leg stabilizer 1.

In reference to FIG. 7 and FIG. 8, the ventilation hole 14 is located between the strap openings 13 and the brace straps 12 to prevent interference from the straps and allow airflow around the knee, mitigating overheating and perspiration.

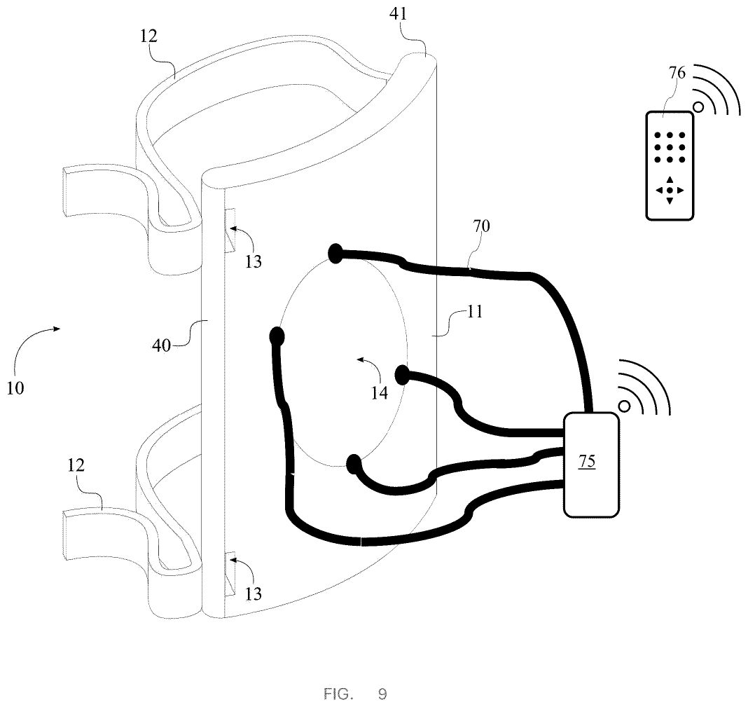

In certain embodiments, as shown in FIG. 9, the knee brace 10 may incorporate multiple medicine tubes 70 positioned around the ventilation hole 14, with a medicine reservoir 75 attached at one end, opposite the connection to the ventilation hole 14. In selected implementations, a remote controller 76 may interface with the medicine reservoir to enable remote dispensing of medicinal fluids into the tubes.

In some embodiments, the medicine reservoir 75 can be attachable and detachable from the knee brace for easy replacement.

In some embodiments, the remote controller 76 is configured with user-friendly controls to dispense specified amounts of medicinal fluids.

In some embodiments, the medicine tubes 70 are manufactured from a material that prevents backflow and contamination of the medicinal fluids.

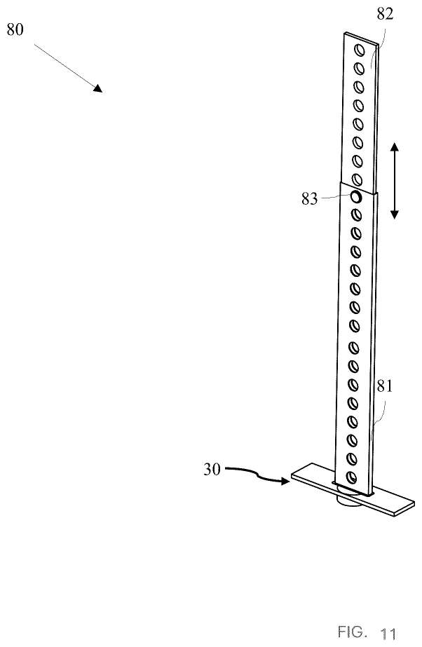

With reference to FIG. 11, the telescopic panel assembly 80 comprises an outer sleeve 81, an inner sleeve 82, and at least one spring-loaded locking mechanism 83. In preferred embodiments, the outer sleeve 81 and inner sleeve 82 are equipped with a series of locking holes that allow the spring-loaded locking mechanism 83 to securely fasten the telescopic panel assembly 80. This assembly 80 is specifically configured to permit length modifications of the walking-aid apparatus 15 as required at various stages during the patient's recovery. The spring-loaded mechanism 83 may be any known mechanism suitable for securing the assembly. After detaching the pair of upper 17 and lower supports 27, the user may extend the length of the walking-aid apparatus 15 by pulling the inner sleeve 82 away from the outer sleeve 81 or reduce the length by inserting the inner sleeve 82 back into the outer sleeve 81.

In some embodiments, the walking-aid apparatus is supported by both a pair of upper supports 17 and a pair of lower supports 27. The lower support 27 may comprise a pair of bent sleeves 28, a pair of elongated sleeves 29, and a replaceable foot 30.

In preferred configurations, the bent sleeves 28 connect adjacently to the elongated sleeves 29, which serve as a bridge connecting the bent sleeves 28 to the replaceable foot 30. The replaceable foot 30 is anchored to the opposing end of the elongated sleeves 29, allowing it to make contact with the ground while supporting the injured leg. The bent sleeves 28 attach to the upper support assembly, opposite the elongated sleeves 29, and are designed to converge, resulting in a singular ground-contacting point with the replaceable foot 30.

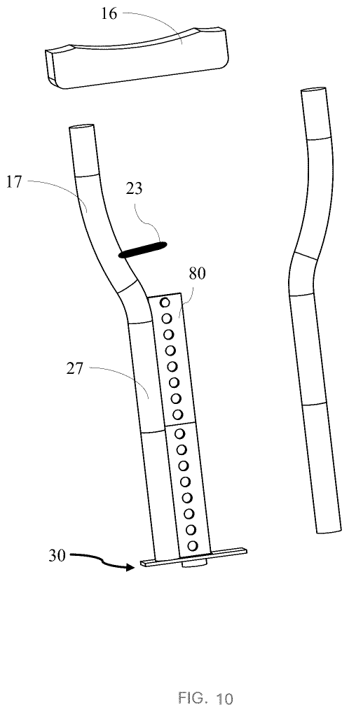

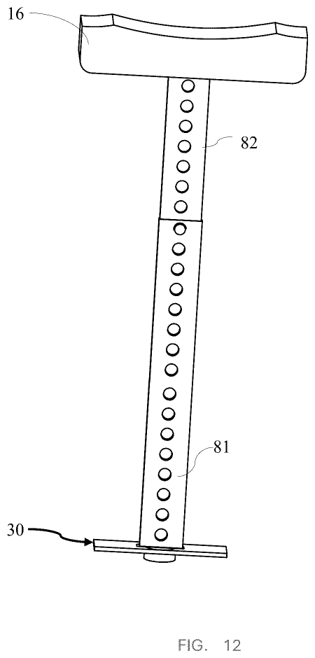

In preferred embodiment, as show in FIG. 10, the pair of the upper supports 17 and the pair of the lower supports 27 of the walking-aid apparatus 15 are configured to be detachable form the telescopic panel assembly 80 so that the telescopic panel assembly 80, as shown in FIG. 11, can be used independently with the first handle attached to the top of the telescopic panel assembly 80 as shown in FIG. 12.

Although the invention has been explained in relation to its preferred embodiment, it is to be understood that many other possible modifications and variations can be made without departing from the spirit and scope of the invention.

What is claimed is:

1. A walking aid system comprising:
a leg stabilizer configured to maintain a user's leg in a straight or bent position, the leg stabilizer including an adjustable angle hinge, a plurality of medial strap openings, and lateral strap openings for securing the user's leg;
a knee brace configured to protect the front, medial, and lateral sides of the knee; and
a walking-aid apparatus comprising a first handle positioned for support under the armpit or in the hand of a user, a second handle offset from the first handle, a telescopic panel assembly, a pair of upper support connected to the first handle and the second handle, and a pair of lower support converging to a singular ground-contacting point, wherein the telescopic panel assembly is positioned in between the pair of lower support.

2. The walking aid system of claim 1, wherein the plurality of stabilizer straps are made from nylon.

3. The walking aid system of claim 1, wherein the leg stabilizer includes a foot plate, a heel plate, a calf plate, and a thigh plate configured to maintain proper alignment of an injured leg.

4. The walking aid system of claim 1, wherein the knee brace further comprises a plurality of brace straps and strap openings for secure attachment to the knee.

5. The walking aid system of claim 1, wherein the upper support assembly of the walking-aid apparatus is configured to be telescopically adjustable in height.

6. The walking aid system of claim 1, wherein the pair of the upper support and the pair of the lower support of the walking-aid apparatus can be detachable form the telescopic panel assembly.

7. The walking aid system of claim 1, further including a locking mechanism to secure the height adjustment of the telescopic panel assembly of the walking aid apparatus.

8. The walking aid system of claim 1, wherein the hinge lock includes a push-button mechanism.

9. The walking aid system of claim 8, wherein adjustable angle hinge is attached to a first arm and a second arm and the hinge lock allows for a 360-degree rotational pivot of the first arm relative to the second arm.

10. The walking aid system of claim 9, wherein the adjustable angle hinge further include a plurality of cogs to maintain position when locked.

11. The walking aid system of claim 10, further comprising a resilient spring integrated into the hinge lock to assist with the locking mechanism.

12. A walking aid system comprising:
a leg stabilizer configured to maintain a user's leg in a straight or bent position, the leg stabilizer including an adjustable angle hinge having a plurality of cogs, a plurality of medial strap openings, and lateral strap openings for securing the user's leg;
a knee brace configured to protect the front, medial, and lateral sides of the knee; the knee brace incorporates at least one medicine tube positioned around a ventilation hole placed on the knee brace, and a medicine reservoir attached at one end of the medicine tube for dispensing medicinal fluids;
a remote controller configured to interface with the medicine reservoir, enabling remote dispensing of medicinal fluids through the medicine tubes; and
a walking-aid apparatus comprising a first handle positioned for support under the armpit or in the hand of a user, a second handle offset from the first handle, a telescopic panel assembly, a pair of upper support connected to the first handle and the second handle, and a pair of lower support converging to a singular ground-contacting point, wherein the telescopic panel assembly is positioned in between the pair of lower support;

13. The walking aid system of claim 12, wherein the medicine reservoir is attachable and detachable from the knee brace for easy replacement.

14. The walking aid system of claim 12, wherein the remote controller is configured with user-friendly controls to dispense specified amounts of medicinal fluids.

15. The walking aid system of claim 12, wherein the medicine tubes are manufactured from a material that prevents backflow and contamination of the medicinal fluids.

16. A walking aid system comprising:
a leg stabilizer configured to maintain a user's leg in a straight or bent position, the leg stabilizer including an adjustable angle hinge, a plurality of medial strap openings, and lateral strap openings for securing the user's leg;
a knee brace configured to protect the front, medial, and lateral sides of the knee;
a walking-aid apparatus comprising a first handle positioned for support under the armpit or in the hand of a user, a second handle offset from the first handle, a telescopic panel assembly, a pair of upper support connected to the first handle and the second handle, and a pair of lower support converging to a singular ground-contacting point, wherein the telescopic panel assembly is positioned in between the pair of lower support; and
a plurality of medicine tubes integrated within the knee brace, allowing for the distribution of medicinal fluids via the remote controller.

20. The walking aid system of claim 19, wherein the medicine tubes are positioned to allow continuous monitoring of fluid levels and ensure timely refills via the remote controller.
